(19) United States
(12) Patent Application Publication
(10) Pub. No.: US 2010/0143490 A1
(43) Pub. Date: Jun. 10, 2010

(54) COMPOSITE MATERIAL

(75) Inventors: Gareth Roberts, Cambridge (GB); Robert Daniels, Cambridge (GB); Xiaobin Zhao, Cambridge (GB); Ian Thompson, Cambridge (GB)

Correspondence Address: BROWDY AND NEIMARK, P.L.L.C.;624 NINTH STREET, NW, SUITE 300, WASHINGTON, DC, 20001-5303, US

(73) Assignees: NOVA THERA LIMITED, CAMBRIDGE (GB); PHARMING GROUP NV, LEIDEN (NL)

(21) Appl. No.: 12/438,385

(22) PCT Filed: Aug. 21, 2007
(86) PCT No.: PCT/GB07/03187

§ 371 Date: Dec. 7, 2009

(30) Foreign Application Priority Data
Aug. 21, 2006 (GB) 0616580.7

(51) Int. Cl. A61K 38/40; A61K 33/42; A61P 17/02; A61P 31/04; A61P 31/12; A61P 19/10; C09D 5/14

(52) U.S. Cl. 424/602; 514/12; 106/15.05

(57) ABSTRACT

The present invention provides a composite material comprising lactoferrin and a bioactive glass. The invention also relates to pharmaceutical compositions containing the composite material. Further aspects of the invention relate to the use of the composite material of the for treating a wound, treating or preventing bacterial or viral infections in a wound, preventing viral transmission, regenerating bone, treating osteoporosis, preventing or alleviating bleeding in a wound, sterilising a wound and/or controlling haemorrhaging.

### BACKGROUND

The present invention relates to a composite material having antibacterial and anti-inflammatory properties which has applications in the field of wound repair, device coatings, viral barriers and bone regeneration.

There is a continuous need in medicine for new anti-bacterial materials and products to limit bacteria and viral diseases spreading in healthcare settings. Such products are required either as treatments for open wounds, coatings for medical devices or to act as barriers to prevent viral transmission.

Lactoferrin is a member of the transferrin family of non-heme iron-binding proteins (Metz-Boutigue et al; Eur J Biochem 1984; 145:659-76). Lactoferrin is found mainly in the external secretions of mucosal epithelia, such as milk, saliva, tears, seminal fluid, sweat, and nasal mucus, as well as in bile, pancreatic fluid, and intestinal secretions in mammals. Lactoferrin is also found in the secondary granules of neutrophils, which are the main source of lactoferrin in plasma as it is secreted upon neutrophil stimulation. Several physiological functions, such as broad-spectrum anti-infective, immunomodulatory and anti-inflammatory activities, have been attributed to lactoferrin (Vorland L H, APMIS 1999; 1 07:971-81; Ward et al; Biochem Cell Biol 2002; 80:95-102).

Lactoferrin is present in exocrine secretions that are commonly exposed to normal flora, such as milk, tears, nasal exudates, saliva, bronchial mucus, gastrointestinal fluids, cervico-vaginal mucus, and seminal fluid, and is thought to play a critical role in host primary defense against infections.

The activity of lactoferrin following a range of delivery methods has been tested against both bacteria and viral challenges.

Lactoferrin exerts both bacteriostatic and bactericidal effects on bacteria by binding the iron needed for bacterial growth or by destabilizing the outer membranes of bacteria (Ward et al: Biochem Cell Biol 2002; 80:95-102).

Lactoferrin and peptides derived from lactoferrin have been shown to inhibit the growth of yeast, filamentous fungi and parasitic protozoa by binding to the lipid bilayer of biological membranes and forming pores, which is ultimately followed by cell lysis (Bellamy et al; Med Microbiol Immunol 1993; 182:97-105; Muller et al; Mycoses 1999; 42 Suppl 2:77-82; Wakabayashi et al; J Med Microbiol 2002; 51:844-50; Wakabayashi et al; Curr Pharm Des 2003; 9:1277-87).

Lactoferrin has also been recognized as a potent inhibitor of various viruses via blockage of viral entry, including rotavirus (Superti et al; Med Microbiol Immunol 1997; 186:83-91), human cytomegalovirus, and human immunodeficiency viruses (Swart et al; Adv Exp Med Biol 1998; 443 :205-13; Semba et al; AIDS 1998; 1 2:331-2), herpes simplex virus (Semba et al; AIDS 1998; 12:331-2), and human hepatitis C virus (Ikeda et al: Virus Res 2000; 66:51-63).

Lactoferrin has also been shown to be a potent modulator of bone cell activity and to increase bone regeneration in vivo, having a stimulatory impact on osteoblasts and an inhibitory effect on osteoclasts (Cornish et al; Endocrinology 145(9): 4366-4374).

Despite the potential application of lactoferrin as an antibacterial and/or antiviral compound for use in topical applications to prevent infections of wounds, across susceptible membranes, as a coating for medical devices or as a stimulant of bone regeneration, there are currently no available technologies which enable the controlled delivery of the active lactoferrin protein to the site of interest.

Accordingly it is an object of the present invention to provide technologies to enable the delivery of lactoferrin in a cost efficient and efficacious manner.

Aspects of the invention are set forth below and in the accompanying claims. For the avoidance of doubt, preferred embodiments apply to all aspects of the invention.

### STATEMENT OF INVENTION

A first aspect of the invention relates to a composite material comprising lactoferrin and a bioactive glass.

A second aspect of the invention relates to a pharmaceutical composition comprising a composite material as described herein and a pharmaceutically acceptable carrier, excipient or diluent.

A third aspect of the invention relates to a wound dressing comprising lactoferrin and a bioactive glass.

A fourth aspect of the invention relates to a medical composite which has applications in the stimulation of bone regeneration, said composite comprising lactoferrin and a bioactive glass.

Further aspects of the invention relate to the use of a composite material as described herein in the preparation of a medicament for treating a wound, treating or preventing a bacterial infection, treating or preventing a viral infection and for preventing viral transmission.

Yet further aspects of the invention relate to methods of treating a wound, methods of treating or preventing a bacterial infection, treating or preventing a viral infection, and preventing viral transmission, using the composite material as described herein.

Another aspect of the invention relates to a coating composition comprising the composite material as described herein and a biocompatible carrier, excipient or diluent.

Yet further aspects of the invention relate to a process for coating a device using the coating composition of the invention, and coated devices prepared by said process. Another aspect of the invention relates to a process for preparing a composite material or composition as described herein.

Finally, another aspect of the invention relates to a composite material comprising lactoferrin and a bioactive glass, for use in medicine.

### DETAILED DESCRIPTION

As mentioned above, a first aspect of the invention relates to a composite material comprising lactoferrin and a bioactive glass.

As used herein, the term “composite material” refers to a %mixture of bioglass and lactoferrin, either alone, or in combination with one or more other components such as fillers, binders, diluents, excipients, carriers and the like.

Bioactive glasses have been used for a number of years as bone void fillers and in the reconstruction of dental or facial bone lesions in maxillofacial surgery. Bioactive glasses have been demonstrated to be reabsorbed, non-toxic in vivo and excreted through the body's natural metabolic pathways. The dissolution products of bioactive glasses have also been demonstrated to stimulate osteoblast cell growth in vitro (Christodoulou et al 2006; J Biomed Mater Res B Appl Biomater, 77(2):431-46). Bioactive glasses can also be formulated to enable the controlled delivery of antibacterial products at the site of application (Bellantone et al; 2002; Antimicrobial Agents and Chemotherapy: 46(6): 1940-1945)

Surprisingly, the present applicant has demonstrated that porous bioactive glasses can be formulated to incorporate lactoferrin onto the surface of the bioactive glass particles (powder) or 3-D structures. The material retains its ability to stimulate fibroblast growth and enables the controlled delivery of lactoferrin to the site of required activity. The bioactive glass/lactoferrin composite can be delivered to the required site as a powder, 3D solid or in an aerosol spray. The rate of delivery and dosage of lactoferrin to the target site can be controlled by altering the porosity of the bioactive glass.

Advantageously, the physical incorporation of lactoferrin into the bioactive glass protects the lactoferrin from metabolism/degradation and enables an extended period of lactoferrin bioactivity at the site of interest.

The novel bioactive glass/lactoferrin composite material of the invention can be used as a wound dressing, spray or device coating to treat or prevent bacterial and/or viral infection as described in more detail below.

As used herein, the term “bioactive glass” refers to an inorganic glass material having an oxide of silicon as its major component and which is capable of bonding with growing tissue when reacted with physiological fluids. Bioactive glasses are well known to those skilled in the art and are disclosed, for example, in “An Introduction to Bioceramics”, L. Hench and J. Wilson, Eds. World Scientific, New Jersey (1993).

The bioactive glasses used in the present invention were derived using the sol-gel method, essentially as described in U.S. Pat. No. 5,074,916.

In one preferred embodiment, the bioactive glass is melt derived. Preferably, for this embodiment, the bioactive glass comprises by approximate weight percent of about 42 to about 52% by weight of silicon dioxide (SiO₂), about 15 to about 25% by weight of sodium oxide (Na₂O), about 15 to about 25% by weight calcium oxide (CaO), and about 1 to about 9% by weight phosphorus oxide (P₂O₅).

In another preferred embodiment, the bioactive glass is sol-gel derived. Preferably, for this embodiment, the bioactive glass comprises by approximate weight percent of about 55 to about 80% by weight of silicon dioxide (SiO₂), from 0 to about 9% by weight of sodium oxide (Na₂O), about 10 to about 40% by weight calcium oxide (CaO), and about 0 to about 8% by weight phosphorus oxide (P₂O₅).

The oxides can be present as solid solutions or mixed oxides, or as mixtures of oxides. CaF₂, B₂O₃, Al₂O₃, MgO, Ag₂O, ZnO and K₂O may also be included in the composition in addition to silicon, sodium, phosphorus and calcium oxides. The preferred range for B₂O₃ is from 0 to about 10% by weight. The preferred range for K₂O is from 0 to about 8% by weight. The preferred range for MgO is from 0 to about 5% by weight. The preferred range for Al₂O₃ is from 0 to about 1.5% by weight. The preferred range for CaF₂ is from 0 to about 12.5% by weight. A preferred range for Ag₂O is from 0 to about 3% by weight. A preferred range for ZnO is from 0 to about 3% by weight.

In the context of the present invention, particularly preferred sol-gel derived bioactive glasses are shown below:

COMPOSITION (MOL %) OF
BIOACTIVE GEL GLASSES

| Designation | SiO₂ | CaO | P₂O₅ |
|---|---|---|---|
| 49S | 50 | 46 | 4 |
| 54S | 55 | 41 | 4 |
| 58S | 60 | 36 | 4 |
| 63S | 65 | 31 | 4 |
| 68S | 70 | 26 | 4 |
| 72S | 75 | 21 | 4 |
| 77S | 80 | 16 | 4 |
| 86S | 90 | 6 | 4 |

In one especially preferred embodiment, the glass is 45S5 Bioglass, which has a composition by weight percentage of approximately 45% SiO₂, 24.5% CaO, 24.5% Na₂O and 6% P₂O₅.

In one highly preferred embodiment of the invention, the bioglass is 58S sol-gel bioglass, as defined in the above table, i.e. the bioglass contains about 60% SiO₂, about 36% CaO and about 4% P₂O₅.

In one highly preferred embodiment of the invention, the bioglass is 70S sol-gel bioglass, i.e. the bioglass contains about 70% SiO₂, and about 30% CaO.

In one highly preferred embodiment, the bioactive glass further comprises a silver salt. Advantageously, the inclusion of a silver salt imparts antibacterial properties into the composite of the invention which helps prevent infection in the area undergoing treatment. Preferably, the silver salt is silver oxide. Further details of silver-containing bioglasses are described in U.S. Pat. No. 6,482,444 (Bellatone et al; assigned to Imperial College Innovations).

In one preferred embodiment, the bioactive glass further comprises about 0.1 to about 12% by weight silver oxide (Ag₂O).

Particulate, non-interlinked bioactive glass is preferred. That is, the glass is preferably in the form of small, discrete particles, rather than a fused matrix of particles or a mesh or fabric (woven or non-woven) of glass fibres. Note that under some conditions the discrete particles of the present invention can tend to cling together because of electrostatic or other forces but are still considered to be non-interlinked. Useful ranges of particle sizes are less than about 1200 microns, typically about 1 to about 1000 microns as measured by SEM or laser light scattering techniques. In one preferred embodiment, the size range of the particles is about 100 to about 800 microns. In a more preferred embodiment of the invention, the size range of the particles is about 300 to about 700 microns. In an alternative preferred embodiment, the size range of the particles is less than about 90 microns.

The bioactive glass is preferably prepared using a sol-gel method. When compared with conventional glass production techniques, there are a number of advantages associated with the sol-gel process: lower processing temperatures, purer and more homogenous materials, good control over the final composition, and tailoring of the surface and pore characteristics of the product.

Sol-gel derived glass is generally prepared by synthesizing an inorganic network by mixing metal alkoxides in solution, followed by hydrolysis, gelation, and low temperature firing (around 200-900° C.) to produce a glass. Sol-gel derived glasses produced in this way are known to have an initial high specific surface area compared with either melt derived glass or porous melt derived glass. The process and types of reactions which typically occur in sol-gel formation are described in more detail in U.S. Pat. No. 6,482,444 (Bellatone et al; assigned to Imperial College Innovations).

In order to incorporate lactoferrin, the bioactive glass used in the present invention is preferably porous. Highly porous bioactive glass has a relatively fast degradation rate and high surface area in comparison to non-porous bioactive glass compositions. Preferably, the pore size is from 0 to about 500 μm, more preferably about 50 μm to about 500 μm, even more preferably about 100 μm to about 400 μm. Preferably, the degree of porosity of the glass is from 0 to about 85%, more preferably about 30% to about 80%, and even more preferably about 40% to about 60%.

Porous bioactive glass can be prepared, for example, by incorporating a leachable substance into the bioactive glass composition, and leaching the substance out of the glass. For example, minute particles of a material capable of being dissolved in a suitable solvent, acid, or base can be mixed with or incorporated into the glass and subsequently leached out. Suitable leachable substances are well known to those of skill in the art and include, for example, sodium chloride and other water-soluble salts. The particle size of the leachable substance is roughly the size of the resulting pore. The relative amount and size of the leachable substance gives rise to the degree of porosity. Alternatively, porosity can be achieved using sintering and/or foaming or by controlling the treatment cycle of glass gels to control the pores and interpores of the material. The porous structure may then be impregnated with lactoferrin.

In one preferred embodiment, the bioactive glass is in the form of a 3-D structure, for example fibres, which may be woven into a mesh or fabric. Continuous fibres can be prepared, for example, by extruding the sol through a spinneret. The fibres can then be aged, dried, and thermally stabilized. Long fibres may be woven into a mesh, short fibres may be combined by mixing them with a degradable adhesive, such as a solution of carboxymethylcellulose (CMC). The resulting material is then heated in a kiln to sinter the material and burn off the binder. Lactoferrin is then incorporated into the 3-D structure, typically by soaking the structure in a lactoferrin-containing solution. Thus, in one preferred embodiment, the composite material of the invention is in the form of a 3-D solid.

In another preferred embodiment of the invention, the composite is in the form of a powder. For this embodiment, the bioactive glass is in the form of small, discrete particles which are typically soaked in a lactoferrin-containing solution.

In another preferred embodiment, the composite material of the invention is in the form of an aerosol spray. Further details on aerosol formulations are described below.

Preferably, the ratio of bioactive glass to lactoferrin in the composite material is from 80-99.5:0.5-20 more preferably from 80-99:1-20.

Pharmaceutical Compositions

A second aspect of the invention relates to a pharmaceutical composition comprising a composite material as described above and a pharmaceutically acceptable carrier, excipient or diluent. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

In one preferred embodiment, the pharmaceutical composition is in the form of a wound dressing.

In another preferred embodiment, the pharmaceutical composition is formulated as an aerosol spray. For example, the composite material of the invention may be formulated as a powder (or as a suspension or solution) and combined with one or more pharmaceutically acceptable solid or liquid inert carriers. Typically, the mixture is packaged in a squeeze bottle or admixed with a pressurized volatile, normally gaseous propellant, e.g. pressurized air, nitrogen, carbon dioxide, dichlorodifluoromethane, propane, argon or neon. Such formulations can be prepared by any of the known means routinely used for making aerosol pharmaceuticals and will be familiar to the skilled artisan.

In another preferred embodiment, the pharmaceutical composition is formulated for topical administration. Suitable carriers, excipients and diluents for topical compositions will be familiar to the skilled artisan.

In one preferred embodiment, the pharmaceutical composition comprises at least one additional pharmaceutical agent. Suitable additional pharmaceutical agents include anti-inflammatory agents, analgesics, such as xylocaine and lidocaine, and antibiotics, such as gentimycin, vanomycin, ciprofloxacin, cefotetan and penicillins. Preferably, the pharmaceutical agent does not adversely affect the antibacterial and/or antiviral performance of the lactoferrin-containing composition.

The pharmaceutical composition may further comprise a biological agent. Suitable biological agents include thrombin, stem cells, collagen, growth factors, such as epidermal growth factor, osteogenin, somatomedin, and the like. The pharmaceutical composition may also comprise bio-absorbable components or a bio-absorbable matrix such as collagen and those described in U.S. Pat. Nos. 4,606,337, 6,056,970, and 6,197,325, which are herein incorporated by reference.

In one preferred embodiment of the invention, the pharmaceutical composition further comprises a procoagulant (also known as a coagulation-promoting agent). As used herein, the term “procoagulant” includes any compound or composition that shifts the enzymatic equilibrium of the biochemical pathway or cascade involved in, or related to, coagulation from a resting state to an activated state.

In one preferred embodiment, the procoagulant is lyophilized to a substrate, such as a piece of gauze or surgical mesh which comprises the composite material of the invention. In another preferred embodiment, the procoagulant and the lactoferrin are lyophilized together.

Preferably, the procoagulant is selected from propyl gallate, gallic acid, isopentyl gallate, lauryl gallate, isobutyl gallate, butyl gallate, pentyl gallate and isopropyl gallate. In one highly preferred embodiment, propyl gallate is used in the form of Hemostatin™, which is available from Analytical Control Systems, Inc. (Fishers, Ind.). The skilled person will appreciate that any composition comprising a procoagulant, such as propyl gallate, gallic acid, or derivatives thereof, may be used in accordance with the present invention so long as the composition lacks any agent, such as heparin or warfarin, which will significantly inhibit clotting. See e.g. U.S. Pat. Nos. 5,700,634, 5,451,509, and 5,709,889, which are herein incorporated by reference.

In another preferred embodiment, the procoagulant is a platelet activating factor.

Preferably, the platelet activating factor is selected from thrombin, epinephrine, adenosine diphosphate, calcium and thromboxane.

In another preferred embodiment, the procoagulant is a cellular component. Preferably, the cellular component is collagen or fibronectin.

Wound Dressing

Another aspect of the invention relates to a wound dressing comprising lactoferrin and a bioactive glass. Advantageously, the wound dressing of the invention enables the quick and even delivery of the composite material to the wound surface which assists in the cessation of bleeding and confers an antibacterial and antiviral environment to the open wound.

As used herein, “dressing” and “bandage” may be used interchangeably to refer to a device that may be used to cover, dress, protect, or heal a wound. As used herein, a “wound” includes damage to any tissue in a living organism. The tissue may be internal, external, or a combination thereof. The tissue may be hard or soft tissue. The wound includes any lesion resulting from an agent, injury, disease, infection or surgical intervention.

In one preferred embodiment, the wound dressing further comprises at least one additional pharmaceutical agent. Suitable additional pharmaceutical agents include anti-inflammatory agents, analgesics, such as xylocaine and lidocaine, and antibiotics, such as gentimycin, vanomycin, ciprofloxacin, cefotetan and penicillins.

The wound dressings may further comprise a biological agent. Suitable biological agents include thrombin, stem cells, collagen, growth factors, such as epidermal growth factor, osteogenin, somatomedin, and the like. The wound dressings may also comprise bio-absorbable components or a bio-absorbable matrix such as collagen and those described in U.S. Pat. Nos. 4,606,337, 6,056,970, and 6,197,325, which are herein incorporated by reference.

The wound dressings of the present invention are useful in the treatment of wounds, haemorrhages, burns and the like. Examples of wounds include those caused by lacerations, punctures, and surgery, such as those resulting from motoring accidents and deep thoracic surgery. The wound dressings of the present invention are particularly useful for treating wounds having a large surface area and wounds that are difficult to suture or cauterize. The wound dressings are also useful for promoting healing of tissue grafts and burns.

Preferably, the wound dressings of the present invention also comprise a procoagulant as defined above in a therapeutic amount. As used herein, a “therapeutic amount” of a procoagulant is an amount that promotes blood coagulation, clot formation, or both. For example, a “therapeutic amount” of propyl gallate typically ranges from about 100 μg/cm² to about 3000 μg/cm², preferably about 250 μg/cm² to about 2000 μg/cm², more preferably about 500 μg/cm² to about 1000 μg/cm² of the surface area of a wound. A person of ordinary skill in the art may readily determine the optimal therapeutic amount of a given procoagulant using routine methods in the art.

In one highly preferred embodiment of the invention, the wound dressing further comprises fibrinogen or fibrin, or a mixture thereof. The fibrinogen, fibrin, or both are preferably mammalian, more preferably, human. Alternatively, the fibrinogen, fibrin, or both may be recombinant. Additionally, fibrin may be used in place of or in combination with fibrinogen. Therefore, fibrinogen, fibrin, or both may be used the dressings and methods of the present invention. However, fibrin is less preferred as it is difficult to work with during bandage preparation. As used herein, the term “fibrinogen” may be used interchangeably with “fibrin”.

It is well known that the amount of fibrinogen on the dressing surface is critical to the performance of fibrinogen dressings. Specifically, more fibrinogen yields a faster clotting time with less bleeding from the wound. However, fibrinogen is expensive and a large amount of fibrinogen on a bandage is difficult to use. Therefore, the present invention provides wound dressings further comprising a procoagulant such as propyl gallate (PG). A fibrinogen-containing bandage in accordance with the present invention comprising a procoagulant may provide substantially the same result as a bandage using a greater amount of fibrinogen alone. The present invention also provides methods of treating a wound comprising apply to the wound a dressing as described herein comprising a procoagulant.

As described herein, blood from wounds treated with a fibrinogen-containing bandage in accordance with the present invention comprising a procoagulant coagulate faster than blood from wounds treated with the bandage alone. Additionally, the amount of clotted blood over wounds treated with a bandage comprising a procoagulant is greater than the amount of clotted blood over wounds treated with a fibrinogen bandage alone. Therefore, the present invention also provides methods of increasing the amount of or rate of coagulation of blood from a wound. The present invention also provides methods for increasing the amount of or rate of clot formation.

Therapeutic Applications

Another aspect of the invention relates to the use of a composite material as described herein in the preparation of a medicament for treating a wound.

Thus, the composite material of the present invention is useful in the treatment of wounds, haemorrhages, burns and the like as described above.

Yet another aspect of the invention relates to the use of a composite material as described herein in the preparation of a medicament for treating or preventing a bacterial infection. Typical bacterial infections include Staphylococcus epidermidis, Staphylococcus aureus and E. Coli. A list of bacterial infections upon which the composite material as described herein can be applied is detailed in Naidu A. S. (2000 (Lactoferrin: Natural: Multifunctional: Antimicrobial: CRC Press LLC, Boca Raton, Fla., US 33431).

Yet another aspect of the invention relates to the use of a composite material as described herein in the preparation of a medicament for treating or preventing a viral infection. Typical viral infections include rotavirus, human cytomegalovirus, human immunodeficiency virus, herpes simplex virus and human hepatitis C virus.

Another aspect of the invention relates to the use of a composite material as described herein in the preparation of a medicament for preventing viral transmission. Preferably, for this embodiment of the invention, the medicament is in the form of a membrane or barrier coated with the composite material of the invention which prevents viral transmission.

In one highly preferred embodiment, the composite material is formulated into a gel or cream to be used as a viral barrier.

In another highly preferred embodiment, the composite material is formulated with one or more silicone oils for topical wound care gels. Such gels are useful in the reduction and/or prevention of scars. More preferably, the gel further comprises silver, thereby providing control against infection.

A further aspect of the invention relates to the use of a composite material as described herein in the preparation of a medicament for preventing or alleviating bleeding in a wound.

Another aspect of the invention relates to the use of a composite material as described herein in the preparation of a medicament for sterilising a wound.

A further aspect of the invention relates to the use of a composite material as described herein in the preparation of a medicament for controlling haemorrhaging.

A further aspect of the invention relates to the use of a composite material as described herein in the preparation of a medicament for regenerating bone and/or treating osteoporosis.

Lactoferrin has been shown to be a potent modulator of bone cell activity and to increase bone regeneration in vivo, having a stimulatory impact on osteoblasts and an inhibitory effect on osteoclasts (Cornish et al; Endocrinology 145(9): 4366-4374). In addition, the dissolution products of bioactive glasses have also been demonstrated to stimulate osteoblast cell growth in vitro (Christodoulou et al; 2006; J Biomed Mater Res B Appl Biomater 77(2):431-46).

Another aspect of the invention includes the preparation of the composite material herein in a formulation suitable for application to bone defects, fractures, lesions and injuries. Suitable formulations include powders and 3-D blocks shaped to fit the target break or lesion.

Another aspect of the invention includes the oral delivery of the composite material to provide a systemic dosage of the composite material for the treatment of bone degenerative diseases including but not limited to osteoporosis.

Yet another aspect of the invention relates to the use of the composite described herein in cosmetic surgery or a cosmetic treatment. In one preferred embodiment, the composite of the invention is included in a dermal filler to control infection and/or extend the duration of the effect of the treatment.

Another aspect of the invention relates to a method of treating or preventing a bacterial infection in a subject, said method comprising administering to the subject a composite material as described herein.

Another aspect of the invention relates to a method of treating or preventing a viral infection in a subject, said method comprising administering to the subject a composite material as described herein.

A further aspect of the invention relates to a method of preventing viral transmisson in a subject, said method comprising administering to the subject a composite material as described herein.

Yet another aspect of the invention relates to a method of preventing or alleviating bleeding in a wound, said method comprising contacting a composite material as described herein with the wound.

A further aspect of the invention relates to a method of sterilising a wound, said method comprising contacting a composite material as described herein with the wound.

A further aspect of the invention relates to a method of controlling haemorrhaging in a subject, said method comprising contacting a composite material according as described herein with the subject.

A further aspect of the invention relates to a method of regenerating bone and/or treating osteoporosis, said method comprising contacting a composite material according as described herein with the subject.

Another aspect of the invention relates to a method of stimulating fibroblast growth in a subject, said method comprising contacting a composite material as described herein with the subject.

Another aspect of the invention relates to a method of stimulating fibroblast growth in a biological sample, said method comprising contacting a composite material as described herein with the biological sample. Preferably, the sample is an in vitro or ex vivo sample.

Coating Composition

Another aspect of the invention relates to coating composition comprising a composite material as described above and a biocompatible carrier, excipient or diluent. As used herein the term “biocompatible” refers to a material that the body generally accepts without a major immune response and that is capable of implantation into biological systems, for example, tissue implantation, without causing excessive fibrosis or rejection reactions.

Preferably, the coating composition further comprises a binder, lubricant, suspending agent, additional coating agent or solubilising agent.

In one preferred embodiment, the coating composition is in the form of a liquid. In another preferred embodiment, the coating composition is in the form of a spray.

Another aspect of the invention relates to a process for coating a device, said process comprising contacting the device with a composite material or a coating composition as described herein. Preferably, the process comprises applying the coating composition (or composite) in liquid form to the surface of the device. Preferably, the coating is applied by dipping, spraying or immersing the device in a liquid coating composition and allowing the coating to dry.

A further aspect of the invention relates to a coated device obtainable by the above process. Preferably, the device is a medical device, for example, the device may be a catheter, stent or an artifical joint or bone replacement.

In one highly preferred embodiment, the medical device is a membrane.

Process

Another aspect of the invention relates to a process for preparing a composite material as described herein, said process comprising contacting bioactive glass with lactoferrin.

In one preferred embodiment, the bioactive glass is in the form of a powder.

In another preferred embodiment, the bioactive glass is in the form of a 3-dimensional solid.

Preferably, the composite material of the invention is prepared by immersing the bioactive glass in a solution comprising lactoferrin. Preferably, the solution is an aqueous solution of lactoferrin.

Preferably, the bioactive glass is immersed in the lactoferrin solution for at least 30 minutes.

Preferably, the ratio of bioactive glass to lactoferrin is from 20-99.99:0.01-80 more preferably from 80-99:1-20.

Preferably, the lactoferrin is in solution at a concentration of about 2 mg/ml to about 20 mg/ml.

Another aspect relates to a process for preparing a coating composition according to the invention, said process comprising contacting a composite material as described above with a biocompatible diluent, excipient or carrier.

A further aspect relates to a process for preparing a pharmaceutical composition according to the invention, said process comprising contacting a composite material as described herein with a pharmaceutically acceptable diluent, excipient or carrier. Preferably, the process involves admixing the composite with the pharmaceutically acceptable diluent, excipient or carrier.

The present invention is further described by way of non-limiting example and with reference to the following figures, wherein:

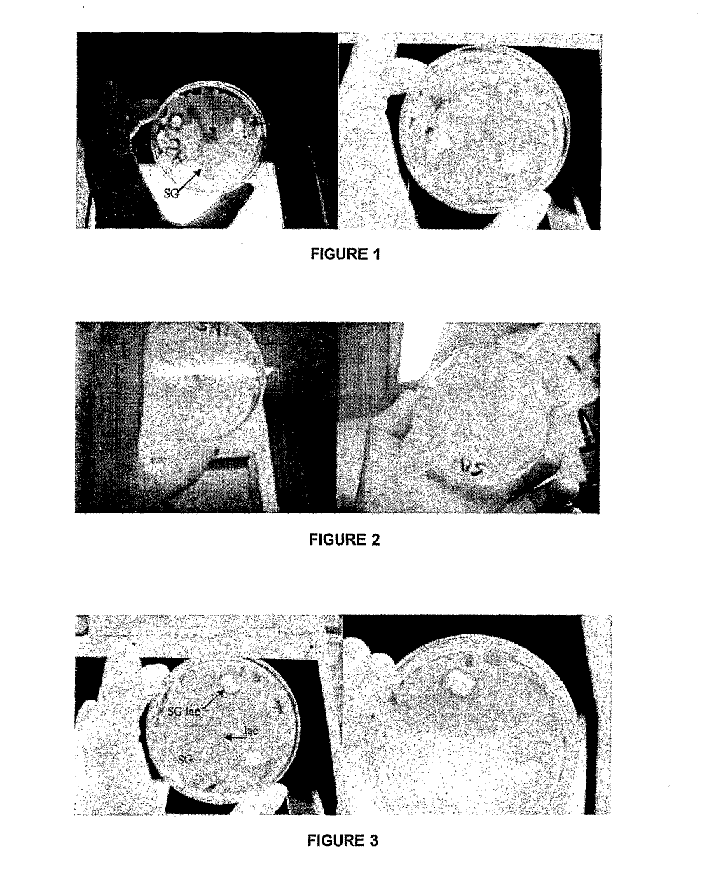

FIG. 1 shows a photograph of the disc diffusion assay of Staphylococcus aureus, demonstrating a zone of inhibition, as indicated as a clear ‘halo’, by lactoferrin-doped sol gel bioactive glass (Sg lac), 100 μg of lactoferrin (lac) after 24 hours incubation.

FIG. 2 shows a photograph of the disc diffusion assay of Staphylococcus aureus, demonstrating inhibition by lactoferrin-doped sol gel bioactive glass (Sg lac) after 48 hours (24 hours on fresh plate) incubation.

FIG. 3 shows a photograph of the disc diffusion assay of Escherichia coli, demonstrating inhibition by lactoferrin-doped sol gel bioactive glass (Sg lac), 100 μg of lactoferrin (lac) after 24 hours incubation.

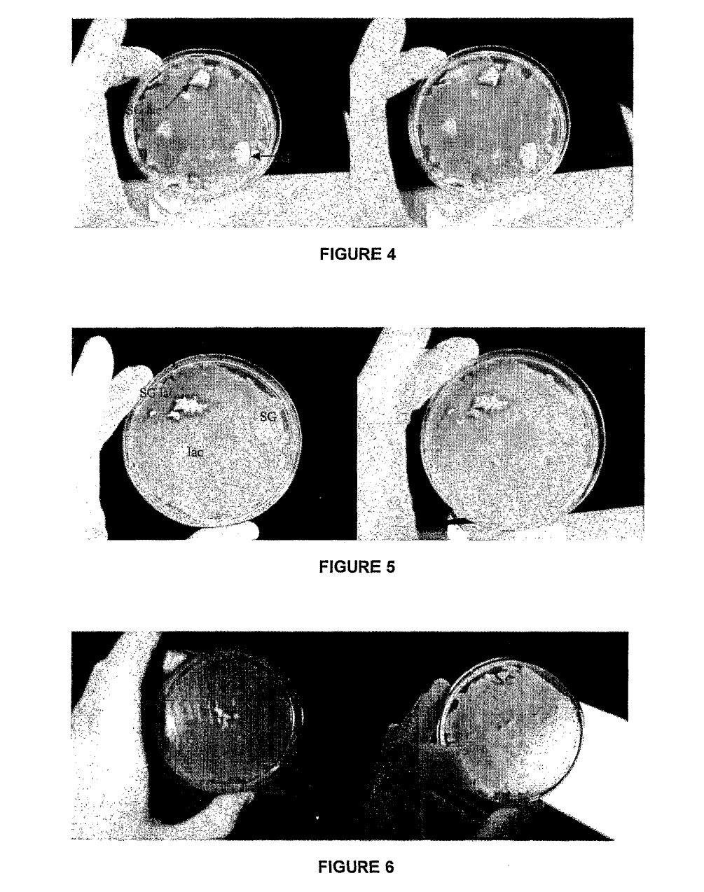

FIG. 4 shows a photograph of the disc diffusion assay of Escherichia coli, demonstrating inhibition by lactoferrin-doped sol gel bioactive glass (Sg lac) after 48 hours (24 hours on fresh plate) incubation.

FIG. 5 shows a photograph of the disc diffusion assay of Staphylococcus epidermidis, demonstrating a zone of inhibition by lactoferrin-doped sol gel bioactive glass (Sg lac), 100 μg of lactoferrin (lac) after 24 hours incubation.

FIG. 6 shows a photograph of the disc diffusion assay of Staphylococcus epidermidis, demonstrating inhibition by lactoferrin-doped sol gel bioactive glass (Sg lac) after 48 hours (24 hours on fresh plate) incubation.

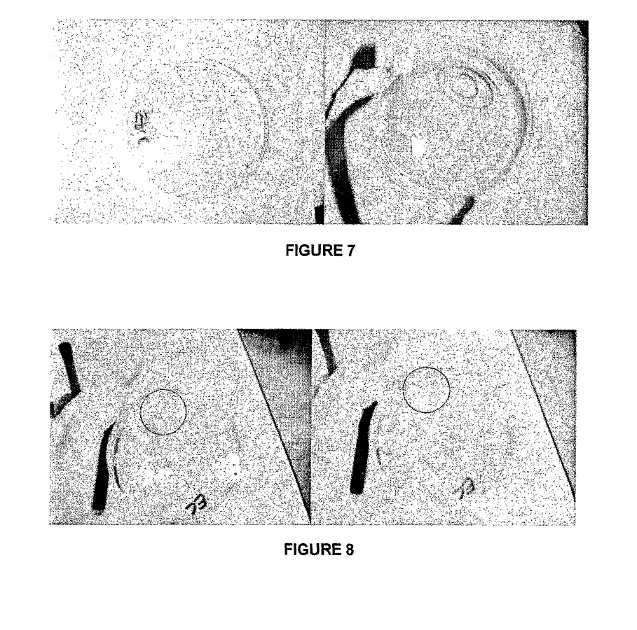

FIG. 7 shows control plates with no bacterial inoculum, containing only phosphate buffer and nutrient broth (50:50).

FIG. 8 shows a photograph of plates used for plate inhibition assay of the first 24 hour incubation step after removal of the sol-gel blocks and further incubation.

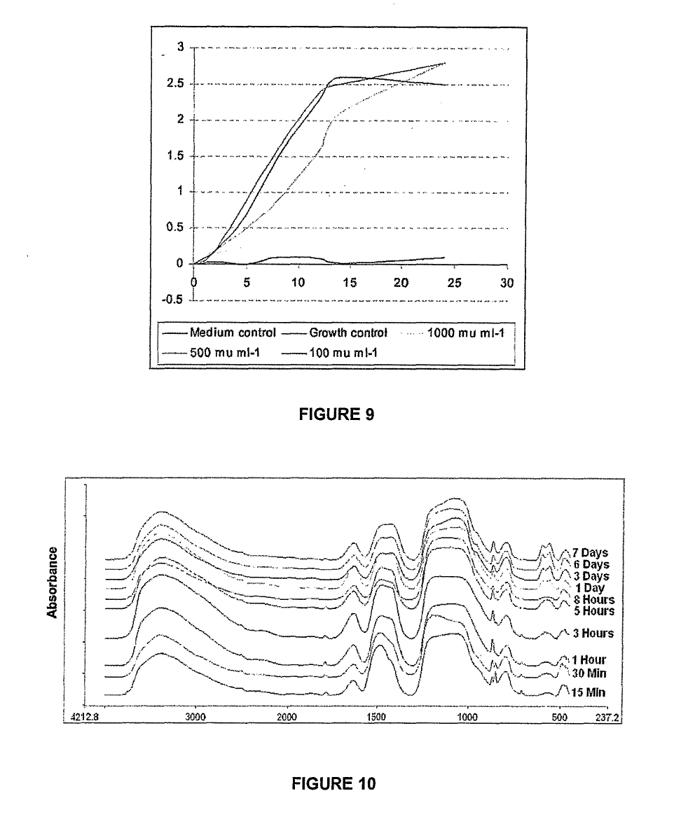

FIG. 9 shows a growth curve for varying amounts of lactoferrin in an S. aureus liquid culture.

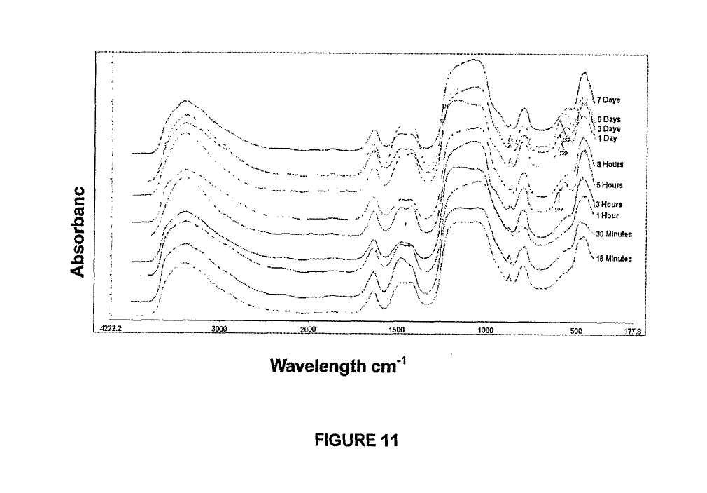

FIG. 10 shows the FTIR spectrum (absorbance versus wavenumber/cm⁻¹) for 58 sol-gel.

FIG. 11 shows the FTIR spectrum (absorbance versus wavenumber/cm⁻¹) for 58 sol-gel+lactoferrin (human milk).

### EXAMPLES

### Example 1

### Assessment of TheraGlass Take-Up of Lactoferrin

Three specimens of TheraGlass (weight 0.7-1.2 g; 58S sol-gel glass) were immersed in a solution of lactoferrin for 30 minutes. The concentration of lactoferrin protein in the solution pre- and post-soaking was measured to assess take-up of lactoferrin by the TheraGlass.

Lactoferrin isolated from human milk was obtained from Sigma-Aldrich (product ref: LO520). Alternatively, lactoferrin may be isolated from bovine milk essentially as described by Naidu A. S., 2000 (Lactoferrin: Natural: Multifunctional: Antimicrobial: CRC Press LLC, Boca Raton, Fla., US 33431).

Bioactive glasses were prepared essentially as described in U.S. Pat. No. 5,074,916.

Protein Concentration of TheraGlass Soaked

| | | Con- | | |
|---|---|---|---|---|
| | Initial | centration | Change in | % Change |
| | concentration | after soaking | concentration | in con- |
| Protein | (mg/ml) | (mg/ml) | (mg/ml) | centration |
| Lactoferrin | 8.1 | 6.05 | 2.55 | 25.31 |

Conclusion
Thera Glass is capable of absorbing lactoferrin protein from solution.

### Example 2

### Analysis of TheraGlass Bioactivity after Incorporation of Lactoferrin FTIR Test Methodology

Lactoferrin from human milk (81.0 g/L) was used in the study. The protein was diluted in 10 ml of ‘water for injection’ to make up the solution to approximately 20 ml. Ten samples of TheraGlass (0.8-1.2 g 58S sol-gel glass) were selected. These samples were tested at different time points: 15, 30 minutes, 1, 3, 5, 8 hours, 1, 3, 6, and 7 days to determine the bioactivity of the glass. Initially, the 10 samples were soaked in the protein solutions for 30 minutes on an orbital shaker at 37° C. After this time period the glass samples were removed and placed in 10 individual sealable containers containing 100 ml Simulated Body Fluid (SBF) essentially as described by Lukito et al 2005 (Materials Letters: 59: 3267-3271). At the individual time points mentioned above, the samples were removed from the SBF solution and placed in a dry glass vial which was transferred to an oven maintained at 37° C.

The reacted dried glass samples were then analysed using Fourier transform infrared spectroscopy (FTIR) (Spectrum 1, FTIR Spectrometer, Perkin Elmer, Buckinghamshire, UK) using methodology described in Warren, L. D., Clark, A. E. & Hench, L. L. “An Investigation of Bioglass Powders: Quality Assurance Test Procedure And Test Criteria”, J. Biomed. Maters. Res. Applied Biomaterials Vol. 23, A2 pp 201-209 (1989).

The controls used in this study were the individual proteins, SBF and dry unreacted TheraGlass. The FTIR spectra for sol-gel 58 (absorbance versus wavenumber/cm⁻¹) for each time point are shown in FIG. 10. The FTIR spectra for sol-gel 58S+lactoferrin (absorbance versus wave number/cm⁻¹) for each time point are shown in FIG. 11.

Results

At 1 day (24 hours immersion in SBF) the P—O bending vibration divided into two peaks between (600-570 cm⁻¹ and 610-600 cm⁻¹) indicating the formation of a crystalline Ca—P layer.

Conclusions
The presence of lactoferrin retards the TheraGlass from producing its HCA layer for up to two days.
The addition of the lactoferrin protein to the TheraGlass reduces the time taken for the glass to be reabsorbed.

### Example 3

### Assessment of Biological Activity of Lactoferrin Following TheraGlass/Lactoferrin Combination

The following experiments were carried out to assess the biological activity of lactoferrin over time following its incorporation into a TheraGlass sol-gel material block. The bacteriostatic and bacteriocidal activity of Lactoferrin was used as an assessment of its biological viability.

Test Methodology

Lactoferrin from human milk (81.0 g/L) was used in the study. The protein was diluted in 10 ml of ‘water for injection’ to make up the solution to approximately 20 ml. Ten samples of TheraGlass (0.8-1.2 g 58S sol-gel glass) were selected. The 10 samples were soaked in the lactoferrin protein solution for 30 minutes on an orbital shaker at 37° C.

Media and Reagents

Nutrient broth (Oxoid) at single and double strength was prepared in deionised water and sterilised by autoclaving at 121° C. for 20 minutes. Media plates were prepared by the addition of 12 g of Agar #1 (Oxoid) per litre of single strength nutrient broth, and poured at 60° C. into sterile disposable plastic Petri dishes according to BSAC protocols, resulting in uniform 4 (−/+0.5) mm media depth. The open media plates were desiccated in a disinfected plate for 30 minutes until gelation. Single colonies of ATCC strains of Staphylococcus aureus, S. epidermidis and Escherichia coli were set up on nutrient agar plates overnight at 37° C. Colonies were subsequently picked and inoculated into single strength nutrient broth and incubated over night.

1 μl of the overnight culture (approx 10¹⁰ colony forming units per ml as determined by spectrophotometry) were diluted in 100 μl of phosphate buffered saline and streaked on fresh nutrient agar plates using a sterile swab (according to BSAC standards). The suspension was allowed to soak in for 5 minutes prior to placement of the experimental samples (10 μl of lactoferrin solution at ˜10 mg/ml, lactoferrin doped sol gel bioactive glass, untreated sol-gel glass). The plates were subsequently placed in an incubator at 37° C. for 24 hours prior to image acquisition, after which the plate inoculation procedure was repeated on a fresh plate and the PBS washed blocks were re-applied on the agar surface facing the same way as previously. After a further 24 hours the blocks were photographed again and the blocks immersed in phosphate buffered saline containing 1 μl of each SYTO-9/propidium iodide (BacLight, bacterial live/dead kit for fluorescent microscopy) and incubated overnight at 4° C. The blocks were subsequently imaged using a Leica SPII Confocal Microscope using a 488 nm excitation line and emission filters for FITC and propidium iodide.

The bacterial inhibition was further assayed by liquid cultures investigating the effect of known amounts of lactoferrin in solution for 24 hours in deionised water and a non-supplemented media only. The bacterial growth curve of Staphylococcus aureus was produced based on photometric absorbance readings at λ=670 nm at several time points over a 24 hour period. Samples were set up in triplicate.

Results

Staphylococcus Aureus: 24 Hours Incubation

FIG. 1 shows a photograph of the disc diffusion assay of Staphylococcus aureus, demonstrating a zone of inhibition, as indicated as a clear ‘halo’, by lactoferrin-doped sol gel bioactive glass (Sg lac), 100 μg of lactoferrin (lac) after 24 hours incubation. No inhibition zones were observed for untreated sol gel bioactive glass (SG).

Staphylococcus Aureus: 48 Hours Incubation

FIG. 2 shows a photograph of the disc diffusion assay of Staphylococcus aureus, demonstrating inhibition by lactoferrin-doped sol gel bioactive glass (Sg lac) after 48 hours (24 hours on fresh plate) incubation. The observed inhibition zone density and diameter was reduced compared to the 24 hour incubation

Escherichia Col: 24 Hours Incubation

FIG. 3 shows a photograph of the disc diffusion assay of Escherichia coli, demonstrating inhibition by lactoferrin-doped sol gel bioactive glass (Sg lac), 100 μg of lactoferrin (lac) after 24 hours incubation. No inhibition zones were observed for untreated sol gel bioactive glass (SG).

Escherichia Col: 48 Hours Incubation

FIG. 4 shows a photograph of the disc diffusion assay of Escherichia coli, demonstrating inhibition by lactoferrin-doped sol gel bioactive glass (Sg lac) after 48 hours (24 hours on fresh plate) incubation. No inhibition zones were observed for untreated sol gel bioactive glass (SG).

Staphylococcus Epidermidis: 24 Hours Incubation

FIG. 5 shows a photograph of the disc diffusion assay of Staphylococcus epidermidis, demonstrating a zone of inhibition by lactoferrin-doped sol gel bioactive glass (Sg lac), 100 μg of lactoferrin (lac) after 24 hours incubation. No inhibition zones were observed for untreated sol gel bioactive glass (SG).

Staphylococcus Epidermidis: 48 Hours Incubation

FIG. 6 shows a photograph of the disc diffusion assay of Staphylococcus epidermidis, demonstrating inhibition by lactoferrin-doped sol gel bioactive glass (Sg lac) after 48 hours (24 hours on fresh plate) incubation. The inhibition zones diameters and densities were significantly reduced compared to the 24 hour incubation.

Controls

FIG. 7 shows plates with no bacterial inoculum, but containing only phosphate buffer and nutrient broth (50:50) to verify the sterility of reagents and techniques.

FIG. 8 shows a photograph of plates used for plate the inhibition assay of the first 24 hour incubation step after removal of the sol-gel blocks and further incubation. The zones of inhibition are still present, but at a higher density of bacteria within. This may indicate, together with the data obtained from 48 hours incubation with the blocks, that a sustained release of lactoferrin takes place when bound to sol-gel blocks.

FIG. 9 shows a growth curve for varying amounts of lactoferrin in an S. aureus liquid culture. More specifically, FIG. 9 shows a growth curve of Staphylococcus aureus in varying amounts of lactoferrin supplementation (0, 100, 500 and 1000 μg ml⁻¹), indicating an initial bacterial growth inhibition for high amounts of lactoferrin, but an increasing gradient after 8 hours incubation, resulting in a final absorption similar to the control material.

Conclusions
The use of lactoferrin in a ‘free suspension’ has a short lived bacterial static effect of 8 hours when used at 1000 μg ml⁻¹. The lactoferrin has little effect when used at 500 μg ml⁻¹.
The lactoferrin has no effect when used at 100 μg ml⁻¹.
The use of lactoferrin with the TheraGlass shows zones of inhibition 3-5 mm distant, radial from the block for 24 hours.
Relocation of the lactoferrin soaked block onto a fresh culture dish showed zones of inhibition 2-4 mm distant, radial from the block for a further 24 hours.
Lactoferrin when absorbed onto a TheraGlass surface has a sustained antimicrobial effect for 48 hours with a dose of 100 μg ml⁻¹.
TheraGlass alone has no antimicrobial effect.
Incorporation of lactoferrin into TheraGlass enables the protection of lactoferrin breakdown and extension of biological activity.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1. A composite material comprising lactoferrin and a bioactive glass.

2. A composite material according to claim 1 wherein the bioactive glass is a sol-gel derived bioactive glass.

3. A composite material according to claim 1 wherein the bioactive glass comprises by approximate weight percent about 55% to about 80% by weight silicon dioxide (SiO₂), from 0% to about 9% by weight sodium oxide (Na₂O), about 10% to about 40% by weight calcium oxide (CaO), and about 0% to about 8% by weight phosphorus oxide (P₂O₅).

4. A composite material according to claim 3, wherein the bioactive glass contains about 60% SiO₂, about 36% CaO and about 4% P₂O₅ by weight.

5. A composite material according to claim 3, wherein the bioactive glass contains about 70% SiO₂ and about 30% CaO.

6. A composite material according to claim 1 which is in the form of a powder or a 3-dimensional solid.

7. (canceled)

8. A composite material according to claim 1 wherein the lactoferrin is derived from milk.

9. (canceled)

10. A pharmaceutical composition comprising lactoferrin, a bioactive glass and a pharmaceutically acceptable carrier, excipient or diluent.

11. (canceled)

12. The pharmaceutical composition according to claim 10 further comprising an anti-inflammatory agent, an analgesic or an antibiotic.

13. The pharmaceutical composition according to claim 10 which further comprises fibrinogen, or fibrin, or a mixture thereof.

14. (canceled)

15. The pharmaceutical composition according to claim 10 further comprising
(a) a procoagulant such as propyl gallate, gallic acid, isopentyl gallate, lauryl gallate, isobutyl gallate, butyl gallate, pentyl gallate and isopropyl gallate,
(b) a platelet activating factor, or
(c) a cellular component.

16. (canceled)

17. The pharmaceutical composition according to claim 10 in the form of a wound dressing.

18. The pharmaceutical composition according to claim 10 formulated as an aerosol spray, or as a gel or cream for topical application.

19.-29. (canceled)

30. A method of treating a wound, comprising the steps of:
(a) preparing a composite material by the process of claim 44, and
(b) contacting the wound with said composite material.

31. A method of treating or preventing a bacterial or viral infection in a subject, comprising the steps of:
(a) preparing a composite material by the process of claim 44, and
(b) administering said composite material to a subject in need thereof.

32.-33. (canceled)

34. A method of stimulating fibroblast growth in a subject, comprising the steps of:
(a) preparing a composite material by the process of claim 44, and
(b) administering said composite material to a subject in need thereof.

35. A method of regenerating bone and/or treating osteoporosis in a subject, comprising the steps of:
(a) preparing a composite material by the process of claim 44, and
(b) administering said composite material to a subject in need thereof.

36. A coating composition comprising
(a) lactoferrin;
(b) a bioactive glass; and
(c) a biocompatible carrier, excipient or diluent.

37. A coating composition according to claim 36 which further comprises a binder, a lubricant, a suspending agent, an additional coating agent or a solubilising agent.

38.-39. (canceled)

40. A process for coating a device, comprising contacting the device with the coating composition according to claim 36.

41. A coated device produced by the process of claim 40.

42. A coated device according to claim 41 which is a medical device such as a catheter, stent or an artificial joint or bone replacement.

43. (canceled)

44. A process for preparing the composite material according to claim 1, comprising contacting the bioactive glass with lactoferrin and allowing the lactoferrin to adhere to said bioactive glass.

45.-46. (canceled)

47. A process according to claim 44 wherein the ratio of bioactive glass to lactoferrin is from 80:20 to 99.5:0.5.

48-51. (canceled)
